(19) United States
(12) Patent Application Publication
(10) Pub. No.: US 2025/0135110 A1
(43) Pub. Date: May 1, 2025

(54) SYSTEM AND METHOD FOR DETECTION AND CONTROL OF A SYRINGE PUMP EMPTY CONDITION

(71) Applicant: CareFusion 303, Inc., San Diego, CA (US)

(72) Inventors: Michael K. WORKMAN, Carlsbad, CA (US); Reza PAIAM, San Diego, CA (US)

(21) Appl. No.: 18/681,847

(22) PCT Filed: Aug. 5, 2022
(86) PCT No.: PCT/US2022/039633

§ 371 Date: Feb. 6, 2024

Related U.S. Application Data
(60) Provisional application No. 63/230,652, filed on Aug. 6, 2021.

(51) Int. Cl. A61M 5/168; A61M 5/145

(52) U.S. Cl. CPC A61M 5/16831; A61M 5/145; A61M 2205/18

(57) ABSTRACT

A trigger condition for entering a syringe empty mode is determined. The trigger condition includes adjusting an operational parameter of an infusion device associated with the syringe to complete a fluid delivery performed by the syringe. The fluid delivery is monitored and, responsive to the fluid delivery satisfying the trigger condition, the infusion device is caused to enter the syringe empty mode. While in the empty mode, a flow rate or threshold associated with the fluid delivery is adjusted to facilitate emptying a fluid from the syringe, and an alert is provided when the threshold associated with the fluid delivery has been satisfied.

### TECHNICAL FIELD

This application relates generally to ensuring that an infusion is completed.

### BACKGROUND

Infusion devices, such as syringe pumps, are used to infuse medical fluids to patients. Due to syringe disposable manufacturing tolerances, it is difficult to measure when a disposable syringe has reached the end of travel during an infusion. Consequently, the syringe drive mechanism can stop prematurely, leaving some medication left in the disposable. In some instances, the syringe drive mechanism may unknowingly bottom out while delivering the medication. In the latter case, the medication in the disposable is administered; however, it may take a significant amount of time for the syringe drive mechanism to determine that it has hit the bottom of the syringe and is no longer delivering medication.

### SUMMARY

If the infusion device improperly detects a syringe is empty before it becomes empty then the patient may not receive all of the prescribed medication. The sooner the device can detect the syringe is not empty, the sooner a clinician can be notified or other measures may be taken so that the patient receives a full dose of the prescribed medicine. Similarly, early detection of a syringe being fully emptied reduces strain on the pump thereby conserving the resources needed to deliver the fluid such as power, pumping motor cycles, and pumping finger wear. There is thus a need for faster detections of reaching the end of a medication delivery, for example, to conserve device resources and ensure delivery of the medication to the patient.

The subject technology provides a mechanism and corresponding algorithm that provides consistent emptying of a syringe, and timely signaling when the syringe becomes empty. In this regard, the subject technology relates to a method for detection and control of a syringe pump empty condition. According to various implementations, the method includes determining a trigger condition for entering a syringe empty mode in which an operational parameter of an infusion device is adjusted to complete a fluid delivery performed by a syringe associated with the infusion device, monitoring the fluid delivery for the trigger condition, responsive to the fluid delivery satisfying the trigger condition, causing the infusion device to enter the syringe empty mode and adjusting the operational parameter to complete the fluid delivery, wherein the operational parameter comprises a flow rate or threshold associated with completing the fluid delivery, detecting that the threshold associated with the fluid delivery has been satisfied, and providing an alert responsive to detecting the threshold is satisfied. Other aspects include corresponding systems, apparatus, and computer program products for implementation of the corresponding method and its features.

The methods and systems described here allow syringe emptying conditions in infusion pumps to be detected much more quickly. Thereby the subject technology ensures delivery of the entire contents of the disposable and, among other benefits described herein, prevents delays in signaling when the syringe is empty.

While the methods and systems disclosed herein are described with regard to syringe pumps, the subject technology is applicable to all infusion pumps. For example, the methods are capable of detecting whether a container volume supplying an infusion fluid (e.g., the medication) is empty. It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1A depicts an example patient care system that includes an infusion device.

FIG. 1B depicts a closer view of a portion of the patient care system shown in FIG. 1A.

FIG. 1C depicts an example of an institutional patient care system of a healthcare organization, according to aspects of the subject technology.

FIG. 2 depicts an example syringe infusion pump, according to aspects of the subject technology.

FIG. 3 depicts a first example fluidic pressure profile as a function of time for detection and control of a syringe pump empty condition, according to various aspects of the subject technology.

FIG. 4 depicts example flow rates that may be employed by the disclosed infusion device, according to various aspects of the subject technology.

FIG. 5 depicts a second example fluidic pressure profile for detection and control of a syringe pump empty condition, according to various aspects of the subject technology.

FIG. 6 depicts an example process for detection and control of a syringe pump empty condition, according to aspects of the subject technology.

FIG. 7 is a conceptual diagram illustrating an example electronic system for detection and control of a syringe pump empty condition, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The time it takes for a syringe drive mechanism to determine that it has hit the bottom of the syringe may be a function of infusion rate and pressure limits. As the drive mechanism continues to push the disposable plunger, the pressure on the force sensor increases until a threshold is reached, signaling that the syringe is empty. This can take minutes to hours depending on the infusion rate and cause significant delays in alarms and line flushing workflows.

As described herein, the subject technology provides a mechanism and corresponding algorithm that provides consistent emptying of a syringe, and ensures timely signaling when the syringe becomes empty. For example, the disclosed system may detect when the syringe is at the end of travel, as determined by a percentage of the disposable size or max critical volume. The system may also detect a pressure (e.g., an upstream pressure or a downstream pressure) in the infusion line or within the syringe. When the plunger of the syringe is in a position near the end of travel and an increase in pressure is sensed, the rate may be increased to more rapidly drive the pressure to a predetermined syringe empty pressure limit.

As an example, in a 50 mL disposable having a +/−1% set compliance, the disclosed algorithm may engage after infusing 99% (e.g., 49.5 mL) from the disposable. The algorithm may then begin to monitor pressure. When an increase in pressure is sensed, the algorithm may cause the pump to increase the rate (while continuously monitoring pressure). In this regard, the pressure may be closely monitored throughout the medication delivery process to ensure that it continues to increase. In some implementations, an amount of syringe travel can be detected and/or limited. Additional or alternative factors that may cause engagement of the disclosed algorithm include: duration of infusion (e.g., amount of elapsed time), percentage of the volume to be infused delivered, infusion rate (e.g., higher rate causes sooner engagement than when pumping at a lower rate; lower rate causes sooner engagement than when pumping a higher rate), drug or drug type (e.g., engage sooner for continuously administered drug than for intermittent drugs; engage sooner for drugs that have short half-life; engage sooner for light sensitive drugs), pending orders for the patient or device (e.g., there is a new disposable waiting to be infused after completion the infusion), or the like.

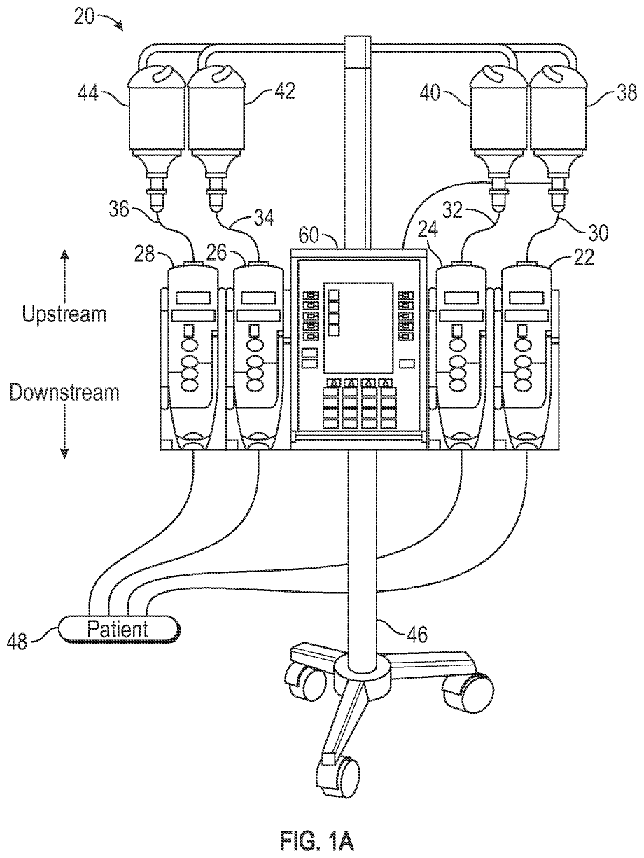

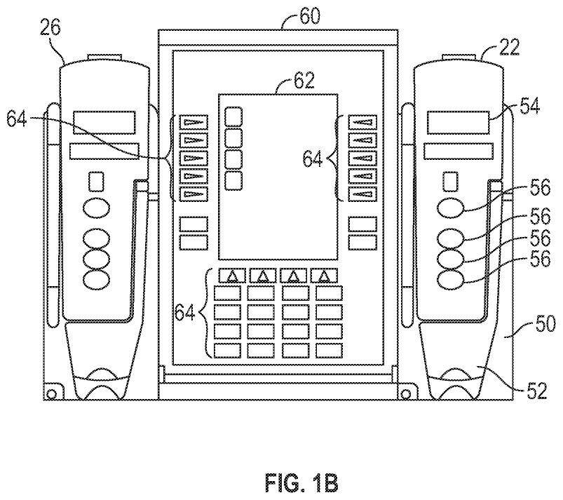

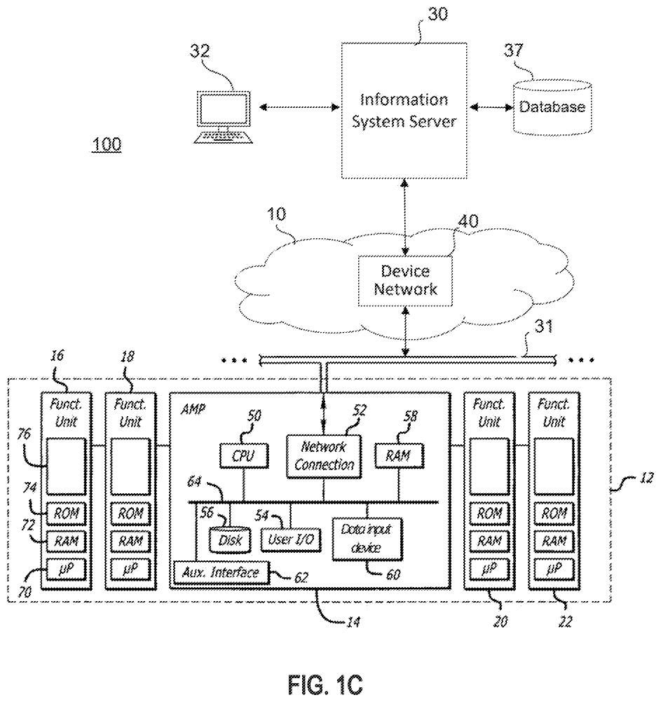

FIG. 1A is an example patient care system, according to various aspects of the subject technology. The patient care system 20 shown in FIG. 1A includes four fluid infusion pumps 22, 24, 26, and 28 each of which is in operative engagement with a respective fluid administration set 30, 32, 34, and 36. Fluid supplies 38, 40, 42, and 44, which may take various forms but in this case are shown as bottles, are inverted and suspended above the pumps. Fluid supplies may also take the form of bags or other types of containers. Both the patient care system 20 and the fluid supplies 38, 40, 42, and 44 are mounted to a roller stand or pole 46. The specific fluid supplies as well as their orientation (e.g., mount location, mount height, mounting type, etc.) within the care area may generate one or more interaction records. The interaction record for a set for example may be generated in part by detecting a scannable code associated with the set or detecting a physical structure on the set that encodes identifying information for the set prior to use.

As shown in the example implementation of FIG. 1A, each administration set 30, 32, 34, and 36 is connected between a respective fluid supply 38, 40, 42, and 44 and the same patient 48 so that the patient may receive the fluids in all the fluid supplies. The administration set may be identified either actively by, for example, scanning by a clinician or passively by, for example, wireless or optical detection of the administration set.

A separate infusion pump 22, 24, 26, and 28 is used to infuse each of the fluids of the fluid supplies into the patient. The infusion pumps are flow control devices that will act on the respective tube or fluid conduit of the fluid administration set to move the fluid from the fluid supply through the conduit to the patient 48. Because individual pumps are used, each can be individually set to the pumping or operating parameters required for infusing the particular medical fluid from the respective fluid supply into the patient at the particular rate prescribed for that fluid by the clinician.

Typically, medical fluid administration sets have more parts than are shown in FIG. 1. Many have check valves, drip chambers, valved ports, connectors, and other devices well known to those skilled in the art. These other devices have not been included in the drawings so as to preserve clarity of illustration.

FIG. 1B is a closer view of a portion of the example patient care system shown in FIG. 1A, according to various aspects of the subject technology. FIG. 1B shows two of the fluid infusion pumps mounted at either side of a programming module, and the displays and control keys of each, with the programming module being capable of programming both infusion pumps. The pump 22 includes a door 50 and a handle 52 that operates to lock the door in a closed position for operation and to unlock and open the door for access to the internal pumping and sensing mechanisms and to load administration sets for the pump. When the door 50 is open, the tube can be connected with the pump 22. When the door 50 is closed, the tube is brought into operating engagement with the pumping mechanism, the upstream and downstream pressure sensors, and the other equipment of the pump. A display 54, such as an LED display, is located in plain view on the door in this embodiment and may be used to visually communicate various information relevant to the pump 22, such as alert indications (e.g., alarm messages). Control keys 56 exist for programming and controlling operations of the infusion pump as desired. In some implementations, the control keys may be omitted and be presented as interactive elements on the display 54 (e.g., touchscreen display). The infusion pump 24 also includes audio alert equipment in the form of a speaker (not shown).

In the embodiment shown in FIG. 1A, a programming module 60 is attached to the left side of the infusion pump 24. Other devices or modules, including another infusion pump, may be attached to the right side of the infusion pump 24 or to the left of the programming module 60, as shown in FIG. 1A. In such a system, each attached pump represents a pump channel of the overall patient care system 20. In one embodiment, the programming module is used to provide an interface between the infusion pump 24 and external devices as well as to provide most of the operator interface for the infusion pump 24. Attention is directed to U.S. Pat. No. 5,713,856 entitled “Modular Patient Care System” to Eggers et al. incorporated herein by reference in which the programming module is described as an advanced interface unit.

Returning to FIG. 1B, the programming module 60 includes a display 62 for visually communicating various information, such as the operating parameters of the pump 24 and alert indications and alert messages. The programming module 60 may also include a speaker to provide audible alerts. In some implementations, the display 62 may be implemented as a touchscreen display. In such implementations, the control keys 64 may be omitted or reduced in number by providing corresponding interactive elements via a graphical user interface presented via the display 62. The programming module 60 may include a communications system (not shown) with which the programming module 60 may communicate with external equipment such as a medical facility server or other computer and with a portable processor, such as a handheld communication device or a laptop-type of computer, or other information device that a clinician may have to transfer information as well as to download drug libraries to a programming module 60 or pump. The communication module may be used to transfer access and interaction information for clinicians encountering the programming module or device coupled therewith (e.g., pump 22 or bar code scanner). The communications system may include one or more of a radio frequency (RF) system, an optical system such as infrared, a BLUETOOTH™ system, or other wired or wireless system. The bar code scanner and communications system may alternatively be included integrally with the infusion pump 24, such as in cases where a programming module is not used, or in addition to one with the programming module 60. Further, information input devices need not be hard-wired to medical instruments, information may be transferred through a wireless connection as well.

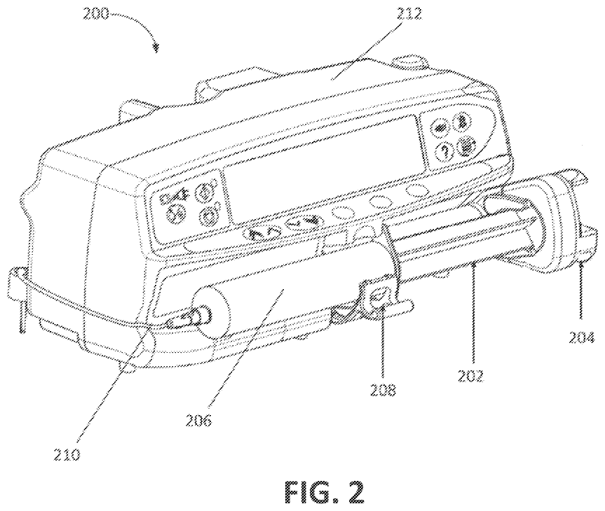

The embodiment shown in FIG. 1B includes a second pump module 26 connected to the programming module 60. As shown in FIG. 1A, more pump modules may be connected. Additionally, other types of modules may be connected to the pump modules or to the programming module such as syringe pump module, as shown in FIG. 2, patient controlled analgesic module, End Tidal CO₂ monitoring module, oximeter monitoring module, or the like.

In some embodiments, the pressure measurements from the upstream and/or downstream pressure sensors are transmitted to a server or other coordination device, and the methods disclosed herein are implemented on the server or other coordination device. For example, more sophisticated and computationally intensive approaches like machine-learning can be implemented on the server (or on a PCU with a larger memory and/or CPU resources). In some embodiments, machine learning is used to identify syringe pump empty conditions based on pressure signals received from the pump.

FIG. 1C depicts an example of an institutional patient care system 100 of a healthcare organization, according to aspects of the subject technology. In FIG. 1C, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a hospital. For example, network 10 of FIG. 1C optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 41 by which patient care devices 12 (and other devices) communicate in accordance with normal operations.

Additionally, institutional patient care system 100 may incorporate a separate information system server 130, the function of which will be described in more detail below. Moreover, although the information system server 130 is shown as a separate server, the functions and programming of the information system server 130 may be incorporated into another computer, if such is desired by engineers designing the institution's information system. Institutional patient care system 100 may further include one or multiple device terminals 132 for connecting and communicating with information system server 130. Device terminals 132 may include personal computers, personal data assistances, mobile devices such as laptops, tablet computers, augmented reality devices, or smartphones, configured with software for communications with information system server 130 via network 10.

Patient care device 12 comprises a system for providing patient care, such as that described in Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control module 14, also referred to as interface unit 14, connected to one or more functional modules 116, 118, 120, 122. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1C to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 116, 118, 120 and 122, and configured to communicate with controller 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 116, 118, 120, 122 are any devices for providing care to a patient or for monitoring patient condition. As shown in FIG. 1C, at least one of functional modules 116, 118, 120, 122 may be an infusion pump module such as an intravenous infusion pump for delivering medication or other fluid to a patient. For the purposes of this discussion, functional module 116 is an infusion pump module. Each of functional modules 118, 120, 122 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Functional module 118, 120 and/or 122 may be a printer, scanner, bar code reader, near-field communication reader, RFID reader, or any other peripheral input, output or input/output device.

Each functional module 116, 118, 120, 122 communicates directly or indirectly with interface unit 14, with interface unit 14 providing overall monitoring and control of device 12. Functional modules 116, 118, 120, 122 may be connected physically and electronically in serial fashion to one or both ends of interface unit 14 as shown in FIG. 1C, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 116, 118, 120, 122 may include module-specific components 76, a microprocessor 70, a volatile memory 72 and a nonvolatile memory 74 for storing information. It should be noted that while four functional modules are shown in FIG. 1C, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 116.

While each functional module may be capable of a least some level of independent operation, interface unit 14 monitors and controls overall operation of device 12. For example, as will be described in more detail below, interface unit 14 provides programming instructions to the functional modules 116, 118, 120, 122 and monitors the status of each module.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction or a combination of both automated and manual interaction. Automated interaction may be continuous or intermittent and may occur through direct network connection 54 (as shown in FIG. 1C), or through RS232 links, MIB systems, RF links such as BLUETOOTH, IR links, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in health information system (HIS) server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication.

FIG. 2 shows an example syringe pump 200 infusion device, according to various aspects of the subject technology. The syringe pump 200 has a drive head that includes a plunger gripper 202 and finger grip release 204. When pressed, the finger grip release 204 causes the fingers of the plunger gripper 202 to separate to accommodate a syringe plunger. A syringe 206 holds a medical fluid to be infused by the syringe pump 200. The syringe 206 is secured by a syringe clamp 208. To deliver the medical fluid, the syringe pump 200 will move the drive head to press the plunger of the syringe 206. The rate is controlled by the syringe pump 200 based on the programmed parameter (e.g., desired rate) and type of syringe.

Syringe pumps do not typically experience any upstream pressure conditions because the fluid to be infused is housed in the syringe 206 and is pushed into an administration set 210 by way of the plunger 202. Downstream pressure conditions can be detected by a force sensor housed in or upon a pump system 212 according to the methods described here, which are readily applied to syringe pumps. The force sensor measures the force exerted by the drive head 204 of the syringe pump on the syringe plunger 202.

In some embodiments, the syringe pump may include a high resolution pressure sensor that interfaces with a pressure disc (not shown) on the syringe administration set. The pressure disc provides a relatively large area in contact with the pressure sensor. This allows the pressure sensor to measure the pressure inside the administration set more directly (not through the syringe plunger head) and with higher resolution and higher accuracy compared with the drive head force sensor. The measurements from this pressure sensor and the drive head force sensor can be used independently or in conjunction with each other to detect an empty condition in a syringe pump.

In an infusion pump, various components that lie in an infusion path such as administration set, cannula, filters, and valves exhibit both resistance and compliance. In normal operation, the pump generates a pressure, termed a working pressure, to overcome the resistance of these and other components in the infusion path. The working pressure depends on a flow rate of the fluid in the infusion path. In particular,

$$Working pressure = Resistance \times Flow rate \qquad ( 1 )$$

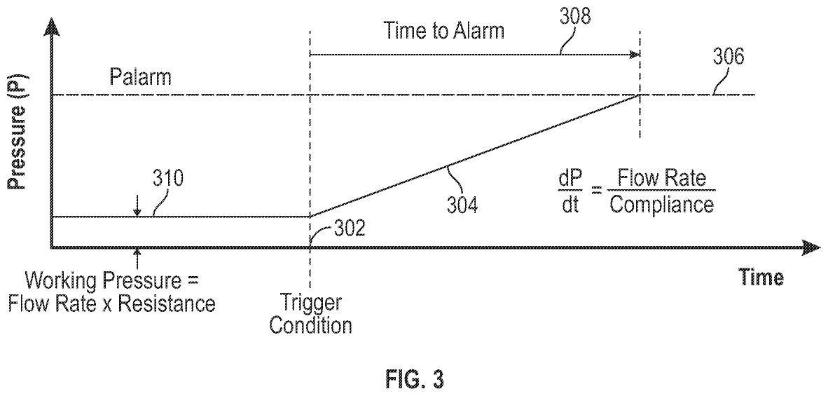

FIG. 3 depicts a first example fluidic pressure profile as a function of time for detection and control of a syringe pump empty condition, according to various aspects of the subject technology. A working pressure 310 is the usual fluidic pressure in the infusion path under normal operation of the infusion pump. The pump may be programmed to monitor pressure within the infusion line, or an amount of fluid already infused or, in some implementations, a travel distance of the plunger. In some implementations, a sensor (described previously) may detect a predetermined force on the drive head or pressure indicative of an empty condition or near empty condition. An empty or near empty condition may be represented by an amount of fluid infused or, in some implementations, based on a measured pressure or force, such as a force on the pump drive head (measured by the drive head force sensor). When a trigger condition occurs, at a time 302, a flow rate and/or fluidic pressure (P) in the infusion path may be increased by the algorithm and the slope 304 monitored. A second threshold 306 (Pₐₗₐᵣₘ) may indicate when the syringe is determined to be emptied. When the detected pressure reaches threshold 306 (Pₐₗₐᵣₘ), the pump may sound an alarm to indicate the syringe is empty.

In general, a rate of pressure increase

$$( \frac{dP}{dt} )$$

depends on the flow rate and the compliance of the administration set, the pump, or syringe in the syringe pump, and other components in the infusion path. Compliance is the inverse of stiffness (which is a measure of the resistance offered by an elastic body to deformation), and can be measured in units of meters per newton.

$$\frac{dP}{dt} = \frac{flow rate}{c o m p l i a n c e} \qquad ( 2 )$$

A time to alarm (TTA) 308, is the time from the onset of trigger condition at the time 302, until the infusion path reaches the fluidic pressure of the set empty threshold 306, Pₐₗₐᵣₘ. According to various implementations, the TTA 308 depends on the set threshold 306 Pₐₗₐᵣₘ and the compliance of the administration set, the pump, or syringe in the syringe pump, and other components in the infusion path.

$$Time of Alarm ( TTA ) = ( {P}_{a l a r m} - working pressure ) \times \frac{c o m p l i a n c e}{flow rate} \qquad ( 3 )$$

According to Equation (3), the TTA 308 increases at lower flow rates and/or for larger compliance values.

The TTA 308 may be reduced by increasing flow rate or pressure acting on the fluid by the delivery mechanism. In some implementations, on the infusion device detecting trigger condition 302, the infusion device may attempt to increase the flow rate, for example, by increasing a pumping speed. The infusion device may then continue to monitor the pressure P and/or pressure change (ΔP) to ensure the syringe is being or has been properly emptied.

While a value of the fluidic pressure generally remains approximately constant (e.g., flat) during delivery of a medication, the pressure rise (ΔP) over a particular time interval is directly proportional to the amount of volume (ΔV) of fluid infused over that interval, i.e.,

$$Δ P = \frac{Δ v}{compliance} \qquad ( 4 )$$

where ΔV=flow rate x time interval of infusion

According to some implementations, the trigger condition 302 and/or empty condition 306 may occur upon the measured pressure (or force) signal behaving differently. For example, trigger condition 302 may be detected when a change in pressure (ΔP) over a particular time interval is detected. The alert condition 306 may be detected upon detecting that pressure P has reached a predetermined limit, plateaus, falls off, or further increases in pressure (ΔP) over a particular time interval (e.g., a logarithmic increase in pressure).

While pressure change is depicted, the subject technology is capable of detecting an end of infusion event my other methods and systems, and pressure may be substituted by a different variable. For example, the trigger condition 302 may be satisfied by a mechanical milestone. For example, the syringe pump 200 may include a laser, camera, or other mechanical sensor to observe the location of the syringe drive head of associated plunger component of the syringe pump 200, and the trigger condition satisfied based on a distance the drive head or other component has moved during the current fluid delivery.

According to some implementations, the system algorithm may monitor pressure P for a predetermined pressure pattern based on the disposable type. For example, each disposable may be subjected to lab/bench testing to estimate/characterize its performance under normal conditions. For example, a particular disposable syringe may be subjected to testing to determine its expected change in pressure (ΔP) when the syringe reaches a predetermined amount of infused fluid. The disposable may be further tested to determine its performance characteristics when used with different infusion sets and/or medications. For the purpose of this disclosure normal operations and/or conditions may include those parameters and/or environmental conditions used during manufacturer testing and characterization process to set a baseline for any variations and/or changes (e.g., ΔP) observed during the testing.

Identifiers for types of syringe disposables, medication and infusion sets, as well as any associated devices disclosed herein, may be stored, for example, in a lookup table or database indexed by the corresponding identifiers. Each identifier, or combination of identifiers, may be associated in the table with a particular operation parameter, or parameters, or pattern of parameters. For example, a particular type of syringe may be tested with a particular infusion set and a pressure curve identified that is representative of the syringe having a remaining amount of solution near 5% of its total volume, and the identified pressure curve indexed in the table by the identifier of the syringe type and/or infusion set type. In this manner, by the system may monitor the pressure and when the pressure exhibits the determined curve, the trigger condition 302 may be satisfied. Additionally or in the alternative, slope 304 may be monitored, and when the slope is matched to the pattern the end of the infusion is reached.

Accordingly, upon set up of an infusion, a clinician may enter various pump parameters, including a syringe type and/or an infusion set type, for example, before initiating the infusion for a patient. In some implementations, the identifiers may be scanned in or automatically received when the corresponding devices are loaded, for example, based on radio frequency identification (RFID) technologies. The infusion device then performs a lookup for the corresponding pressure indictors for trigger condition 302, pressure curve 304, and/or alert condition 306, which may then be used by the disclosed algorithm to perform the various detections disclosed herein.

In some implementations, an alert may be triggered on reaching trigger condition 302 and/or alert condition 306. The alert may be displayed, for example, by the infusion device. In this regard, the alert may indicate the syringe is empty or near empty, depending on the condition satisfied. The alarm may be a human perceivable indication such as via a user interface, light, sound, or haptic feedback. The display of the infusion device may display the alert. Additionally or in the alternative, the infusion device may transmit an alert message via the server to a remote receiver such as nursing station in a hospital.

In some implementations, on reaching trigger condition 302 and/or the alert condition 306, the infusion device may additionally or alternatively adjust the operation of one or more physical elements of the system. For example, the infusion device may disable power to the motor driving the pump, increase pump speed, initiate a back-off (e.g., reverse the syringe pump drive head to pull the plunger back), or the like. In some implementations, the infusion device may adjust operation of a second infusion pump (e.g., infusion module). For example, the algorithm may cause a second syringe to be activated to flush a medication or fluid (e.g., saline) into a common connector (e.g., a y-line).

In some implementations, upon reaching a trigger condition 302, the algorithm may automatically change the alert limit 306 based on various other factors. For example, the algorithm may begin to monitor the change in pressure (ΔP) and determine the change is not sufficient to reach a limit 306 within a predetermined period of time. To ensure timely emptying of the disposable, the algorithm may then speed up pumping to increase the flow rate to reach limit 306 within a desired (e.g., predetermined) period of time, or for a minimal time given safe conditions for the given infusion (e.g., determined by operating limits of the infusion device and/or infusion guidelines set by the healthcare organization). In some implementations, the limit 306 may be lowered, for example, so that an end of infusion condition 306 is detected earlier than originally programmed, and the clinician notified (e.g., by any of the previously described notification methods). In some implementations, the algorithm (e.g., on detecting an insufficient pressure change) may halt pumping altogether and activate the alert.

In some implementations, the algorithm may activate a predetermined flow profile, such as a flow rate profile described in co-pending U.S. application Ser. No. 17/240,857, filed Apr. 26, 2021, incorporated herein by reference for all purposes.

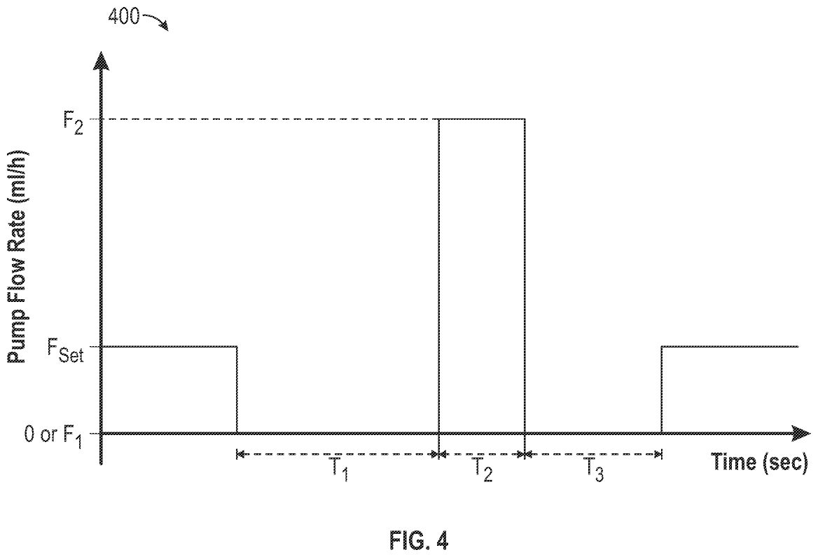

FIG. 4 depicts an example flow profile, including example flow rates F₁ and F₂ that may be employed by the disclosed infusion device, according to various aspects of the subject technology. A pressure signature corresponding a predetermined change in pressure may be stored and activated for ensuring that a given disposable is emptied. In the depicted pump flow rate profile 400, the pump flow rate is set at F₁ (or 0 ml/hour) during a first time interval T1. During a second time interview T₂, the pump flow rate is set at F₂, and during a third time interval T₃, the pump flow rate is set at F₁ again. In general, the pump may set a third flow rate F₃ during the third time interval T₃. In some implementations, the third flow rate F₃ equals to the first flow rate F₁. In some implementations, the first flow rate F₁ (and the third flow rate F₃) is 0 ml/hour. The pump flow rate profile 400 operates at a set flow rate Fₛₑₜ prior to the first time interval T₁ and after the third time interval T₃.

A value of the fluidic pressure may remain approximately constant (e.g., flat) within each of these intervals: an approximately constant value of P₁ over the first time interval T₁ when the system operates at the first flow rate (F₁), and an approximately constant value of P₂ over the second time interval T₂ when the system operates at the second flow rate (F₂).

In some embodiments, the trigger for the pump activating (e.g., generating) an adjusted flow rate (e.g., increased or decreased) of the flow rate profile is the detection of a rising slope for downstream fluidic pressures or the detection of a falling slope for upstream pressures, both which may be indicative of an end of infusion condition. The profile may determine by what amount a flow rate is increased (or lowered), depending on the state of the syringe (e.g., near empty or empty). When the fluid of the syringe is determined to be near empty (e.g., as indicated by a threshold condition 302 being satisfied) the profile may increase the pressure and/or flow rate. When the fluid of the syringe is determined to be empty then the profile may decrease the pressure and/or flow rate.

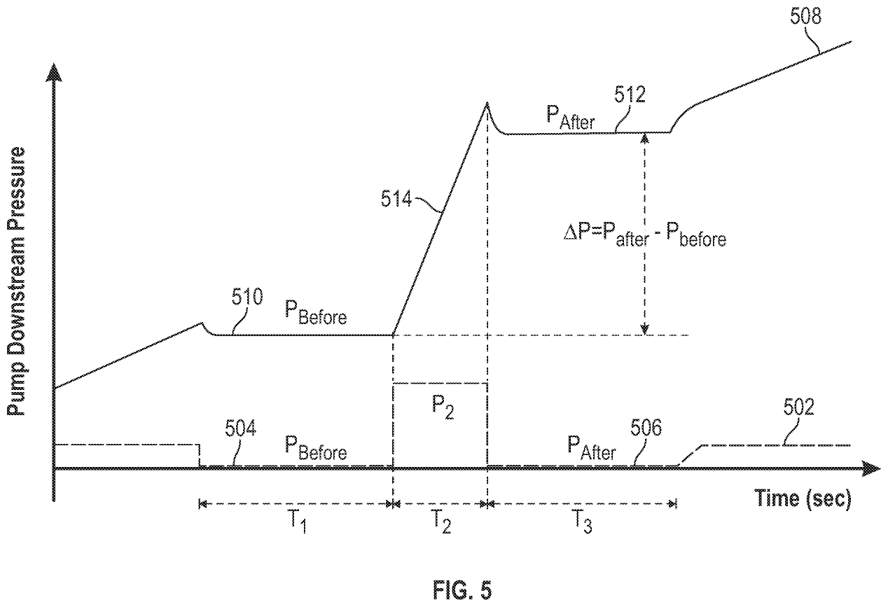

FIG. 5 depicts a second example fluidic pressure profile for detection and control of a syringe pump empty condition, according to various aspects of the subject technology. A value of a pressure 504 before (e.g., P_{before}) and a value of a pressure 506 after (e.g., P_{after}) the time interval T₂ may be the same. The change in pressure between time intervals T₁ and T₂ (e.g., ΔP=P₂−P_{before}) may be given by:

$$( {P}_{2} - {P}_{before} ) = flow rate \times resistance \qquad ( 7 )$$

where resistance refers to the resistance introduced by the administration set, the cannula, the subject's vein, valves, and other components along the infusion path.

In some implementations, T₁ may be representative of normal infusion conditions and a normal pressure 510, prior to a trigger condition. On a trigger condition (indicating that the pump is nearing an empty condition), the pressure may be programmed to rise 514 (according to the given profile) during T₂. According to various implementations, the rise 514 may be due to the pump increasing the flow rate F₂ to ensure emptying of the syringe. The pressure change, ΔP, is controlled by adjusting F₂ and T₂, where F₂×T₂=ΔV, the volume infused during time interval T₂. ΔV is typically a few microliters. The pressure curve 512 after (P_{after}) may represent a steady pressure state detected while emptying the syringe.

The example curve 508 illustrates an example downstream fluidic pressure (or force) profile in the infusion path indicative of an empty condition. For example, the system may detect a second rise 508 at the syringe drive head due to the drive head being fully driven to its maximum limit. In some instances the detected pressure rise 518 may be sharp or logarithmic. In some implementations (e.g., where a line pressure is monitored), pressure change 508 may fall off or become negative, indicating the end of the fluid flow. The pressure curves 502 and 508 shown in FIG. 5 are for demonstration purposes and are not drawn to scale.

The profile shown in FIG. 5 illustrates one example of an algorithm that can be engaged to efficiently identify the end of a container. Additional or alternative detection algorithms may be engaged to identify the end of a container according to the features described. Understanding when to start an end of container detection algorithm can conserve pump resources by deferring the sensor collection and processing of sensor readings. Furthermore, some detection algorithms disrupt flow continuity to identify the end of a container. Understanding when to start an end of container detection algorithm can minimize such disruptions.

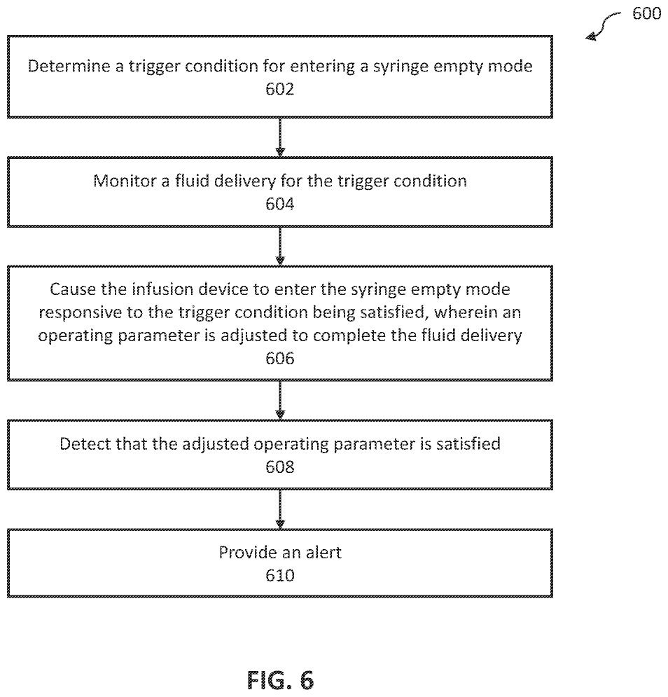

FIG. 6 depicts an example process 600 for detection and control of a syringe pump empty condition, according to aspects of the subject technology. For explanatory purposes, the various blocks of example process 600 are described herein with reference to FIGS. 1-5, and the components and/or processes described herein. The one or more of the blocks of process 600 may be implemented, for example, by one or more computing devices including, for example, medical device 12. In some implementations, one or more of the blocks may be implemented based on one or more machine learning algorithms. In some implementations, one or more of the blocks may be implemented apart from other blocks, and by one or more different processors or devices. Further for explanatory purposes, the blocks of example process 600 are described as occurring in serial, or linearly. However, multiple blocks of example process 600 may occur in parallel. In addition, the blocks of example process 600 need not be performed in the order shown and/or one or more of the blocks of example process 600 need not be performed.

In the depicted example, a medical device 12 monitors, various delivery conditions associated with the administration of a medication to the patient. In this regard, the medical device 12 receives one or more inputs from a clinician regarding the setup of the infusion. These inputs may include operational parameters from which the delivery conditions may be derived, including an identification of the infusion set used, type of medication and/or a medication order, and various physiological parameters of the patient (e.g., height, weight, blood pressure). One or more of these parameters may be manually entered at a user interface of the device. One or more of these parameters may be scanned. And, one or more of these features may be automatically measured by the device 12. For example, when a syringe for a syringe pump is loaded, the infusion device 12 may automatically detect the type of syringe and automatically load a pressure curve for determining an empty condition.

In the depicted example, the infusion device 12 determines a trigger condition for entering a syringe empty mode (602). The syringe empty mode may be a mode in which an operational parameter of the infusion device is automatically adjusted (e.g., by a processor associated with infusion device 12) to complete a fluid delivery performed by the syringe.

According to various implementations, the trigger condition is determined based on a characteristic of a syringe coupled to the infusion device. In some implementations, the characteristic may comprise data provided by an external source. As described previously, the type of syringe, medication, infusion set, and the like may be characterized during manufacturer testing and data for determining the trigger source stored in a database. The infusion device may then receive an identifier associated with the type of syringe used in the current fluid delivery, and perform a parameter lookup based on the identifier to obtain the trigger condition. According to some implementations, the trigger condition involves one or more predetermined thresholds that must be satisfied by a performance characteristic of the current infusion. For example, threshold may include a pressure threshold, amount of fluid infused, or a distance in which the plunger of the syringe has moved during the current infusion. Additionally or in the alternative, the trigger condition may be determined based on a rate of the fluid delivery or historical data for one or more other fluid deliveries.

The infusion device proceeds to monitor the fluid delivery for the trigger condition (604). In some implementations, the trigger condition may be a predetermined pressure threshold. In this regard, a real-time delivery pressure associated with the fluid delivery may be monitored (e.g., at periodic intervals) and the trigger condition satisfied when the real-time delivery pressure reaches the predetermined pressure threshold. In some implementations, the real-time delivery pressure is monitored according to a first frequency during normal operations (e.g., before the fluid delivery satisfies the trigger condition) and the rate of monitoring increased to a second frequency responsive to the infusion device detecting the trigger condition and entering the syringe empty mode. For example, some pumps may monitor infusions at a slower, periodic rate (e.g., taking a measurement every minute or so). On entering the empty mode, the infusion device may switch to a continuous monitoring scheme wherein the infusion is monitored much faster (e.g., at 1-10 second intervals). In some implementations, the trigger condition is a threshold amount of pressure detected in the fluid line, or a threshold amount of force on the drive mechanism and/or plunger.

In some implementations, an amount of fluid delivered by the syringe to a patient is periodically determined and the trigger condition is satisfied when a predetermined amount of fluid is delivered to the patient. For example, the trigger condition may be a percentage of the fluid infused or a time span of the infusion, which may be further based on the rate of infusion (e.g., amount of time at a given rate). As described previously, the trigger condition may be indicated by matching a real-time pressure to a pressure curve. In this regard, the pressure curve may be representative of how a measured pressure (e.g., downstream of the syringe) changes during a predetermined period at an end of the fluid delivery. For example, the trigger condition may be triggered based on a fluid pressure measured downstream of the syringe satisfying a predetermined pressure curve, and the pressure curve may be representative of how the pressure measured downstream of the syringe changes during a predetermined period at an end of the fluid delivery.

In some implementations, a motion the plunger is monitored and the trigger condition is satisfied when the plunger has moved a predetermined distance. The infusion device 12 or an associated monitoring system operatively connected to the device may employ a camera or an optical sensor to monitor the plunger's motion. For example, the camera or the optical sensor may be coupled to the syringe pump and may detect when a marker on the plunger reaches a sensor location, thereby indicating that the plunger reached the predetermined distance. The camera may employ image recognition techniques, while the optical senor may recognize a light difference based on the marker.

According to various implementations, the trigger condition may be a single condition or a combination of conditions. For example, the trigger condition may be satisfied upon satisfying one or more of the real-time delivery pressure satisfying a predetermined pressure, a predetermined amount of fluid being delivered to the patient, the motion of the plunger reaching a predetermined distance, and/or matching a real-time pressure to a pressure curve, etc. In some implementations, a combination of criteria may need to be met to satisfy the trigger condition. In some implementations, more than one conditions may be cascaded such that one condition is satisfied before another is checked.

Responsive to the fluid delivery satisfying the trigger condition, an algorithm causes the infusion device to enter the syringe empty mode (606). In the empty mode, the operational parameter (e.g., introduced in step 602) is adjusted to ensure that the fluid delivery is completed. According to various implementations, the adjusted operational parameter includes a flow rate or a threshold associated with the fluid delivery (e.g., the system may adjust the original threshold to facilitate emptying a fluid from the syringe). In some implementations, when adjusting the threshold, the adjusted threshold may be a limit associated with the fluid delivery being complete. For example, the threshold limit may be a threshold amount of fluid to be delivered, threshold amount of time for the delivery, etc. In some implementations, the flow rate is adjusted, or increased, by activating a second pump, as discussed below.

Additionally or in the alternative, the infusion device 12 may be caused to initiate a mechanical action. In some implementations, responsive to the infusion device entering the syringe empty mode, a second infusion pump may be caused to start another infusion to the same patient. The second pump may be configured to infuse a fluid (e.g., another medication) using a second IV line connected to the primary IV line by way of a y-line adapter. When the trigger condition is detected, the algorithm (e.g., operating on control unit 14) may automatically activate the second pump. According to some implementations, the second infusion may be initiated to flush the primary fluid line (e.g., with a saline solution) which also provides the fluid delivery from the primary/first pump. In some implementations, the second infusion may be another medication (e.g., the same or different medication) which is started to provide a continuous infusion.

As an example, an infusion device may include multiple functional modules 116, 118, 120, 122, one of which is the first pump and one of which is the second pump. In some implementations, the second pump may be a separate unit, managed by a separate control unit 14. Both pumps may be syringe pumps, or different pumps. For example, the first pump may be a syringe pump and the second pump may a large volume pump. The various possible configurations illustrate the applicability of the subject technology to pumps other than infusion pumps.

In some implementations, adjusting the flow rate or threshold associated with the fluid delivery comprises increasing a speed at which the plunger moves to purge the fluid from the syringe, and facilitates emptying the syringe.

The algorithm detects that the adjusted operating parameter (e.g., the adjusted flow rate or adjusted threshold) has been satisfied (608) and, responsive to detecting the adjusted parameter is satisfied, causes an alert to be provided (610). In some implementations, a medical device 12 may be configured to produce audible or visual alerts when a threshold is reached. The alert may be displayed by the infusion device or on a display screen associated with the infusion device. In some implementations, the alert may include providing for an option to adjust a parameter. For example, the medical device may prompt a user to select whether to terminate the infusion or start a new infusion. In some implementations, under an alert condition the device may be prevented from administering or providing further medications until the alert has been acknowledged. Acknowledgement may include identifying a clinician authorized to the medical device by way of the clinician scanning a badge, and the clinician manually dismissing the alert by way of a manual input at the medical device or by way of a computing device connected to the medical device (e.g., over a network). Accordingly, if the parameter affecting the alert is adjusted and/or corrected, the alert may be prevented and the device.

The foregoing process provides multiple benefits including, but not limited to, ensuring the patient receives all of the prescribed medicine. Moreover, early detection of a syringe being fully emptied reduces strain on the pump thereby conserving the resources needed to deliver the fluid such as power, pumping motor cycles, and pumping finger wear. Indeed, many infusion pumps are battery powered and power limited. In such power constrained systems, the life of a battery may be extended by preventing an infusion from running needlessly when no fluid is being pumped. The system detects the threshold trigger condition and initiates rapid emptying of the syringe or other remedial remedies (such as reducing an end of delivery threshold) to ensure the infusion is timely completed.

Many of the above-described example process 600, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

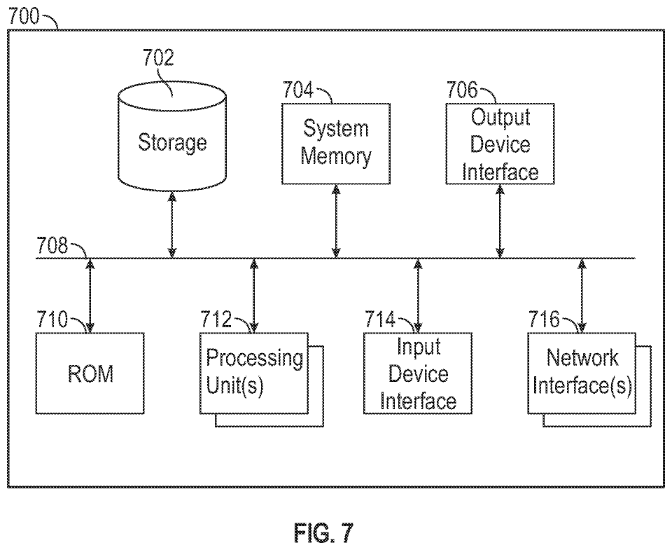

FIG. 7 is a conceptual diagram illustrating an example electronic system 700 for detection and control of a syringe pump empty condition, according to aspects of the subject technology. Electronic system 700 may be a computing device for execution of software associated with one or more portions or steps of method 700, or components and methods provided by FIGS. 1-6, including but not limited to computing hardware within patient care device 12, or syringe pump 200, and/or any computing devices or associated terminals disclosed herein. In this regard, electronic system 700 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 700 may include various types of computer readable media and interfaces for various other types of computer readable media. In the depicted example, electronic system 700 includes a bus 708, processing unit(s) 712, a system memory 704, a read-only memory (ROM) 710, a permanent storage device 702, an input device interface 714, an output device interface 706, and one or more network interfaces 716. In some implementations, electronic system 700 may include or be integrated with other computing devices or circuitry for operation of the various components and methods previously described.

Bus 708 collectively represents all system, peripheral, and chipset buses that communicatively connect the numerous internal devices of electronic system 700. For instance, bus 408 communicatively connects processing unit(s) 712 with ROM 710, system memory 704, and permanent storage device 702.

From these various memory units, processing unit(s) 712 retrieves instructions to execute and data to process in order to execute the processes of the subject disclosure. The processing unit(s) can be a single processor or a multi-core processor in different implementations.

ROM 710 stores static data and instructions that are needed by processing unit(s) 712 and other modules of the electronic system. Permanent storage device 702, on the other hand, is a read-and-write memory device. This device is a non-volatile memory unit that stores instructions and data even when electronic system 700 is off. Some implementations of the subject disclosure use a mass-storage device (such as a magnetic or optical disk and its corresponding disk drive) as permanent storage device 702.

Other implementations use a removable storage device (such as a floppy disk, flash drive, and its corresponding disk drive) as permanent storage device 702. Like permanent storage device 702, system memory 704 is a read-and-write memory device. However, unlike storage device 702, system memory 704 is a volatile read-and-write memory, such a random access memory. System memory 704 stores some of the instructions and data that the processor needs at runtime. In some implementations, the processes of the subject disclosure are stored in system memory 704, permanent storage device 702, and/or ROM 710. From these various memory units, processing unit(s) 712 retrieves instructions to execute and data to process in order to execute the processes of some implementations.

Bus 708 also connects to input and output device interfaces 714 and 706. Input device interface 714 enables the user to communicate information and select commands to the electronic system. Input devices used with input device interface 714 include, e.g., alphanumeric keyboards and pointing devices (also called “cursor control devices”). Output device interfaces 706 enables, e.g., the display of images generated by the electronic system 700. Output devices used with output device interface 706 include, e.g., printers and display devices, such as cathode ray tubes (CRT) or liquid crystal displays (LCD). Some implementations include devices such as a touchscreen that functions as both input and output devices.

Also, as shown in FIG. 7, bus 708 also couples electronic system 700 to a network (not shown) through network interfaces 716. Network interfaces 716 may include, e.g., a wireless access point (e.g., Bluetooth or WiFi) or radio circuitry for connecting to a wireless access point. Network interfaces 716 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 700 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification
Clause 1. A method for detection and control of a syringe pump empty condition, comprising: determining a trigger condition for entering a syringe empty mode in which an operational parameter of an infusion device is adjusted to complete a fluid delivery performed by a syringe associated with the infusion device; monitoring the fluid delivery for the trigger condition; responsive to the fluid delivery satisfying the trigger condition, causing the infusion device to enter the syringe empty mode and adjusting the operational parameter to complete the fluid delivery, wherein the adjusted operational parameter comprises a flow rate or a threshold associated with completing the fluid delivery; detecting that the adjusted operational parameter has been satisfied; and providing an alert responsive to detecting the threshold is satisfied.
Clause 2. The method of Clause 1, further comprising: monitoring a real-time delivery pressure associated with the fluid delivery; and wherein the trigger condition is satisfied based on the real-time delivery pressure satisfying a predetermined pressure.
Clause 3. The method of Clause 2, wherein the real-time delivery pressure is monitored according to a first frequency before the fluid delivery satisfying the trigger condition and increased to a second frequency responsive to the infusion device entering the syringe empty mode.
Clause 4. The method of any one of Clauses 1 through 3, further comprising: determining an amount of fluid delivered by the syringe to a patient; and wherein the trigger condition is satisfied based on a predetermined amount of fluid being delivered to the patient.
Clause 5. The method of any one of Clauses 1 through 4, wherein the syringe comprises a plunger, the method further comprising: monitoring a motion the plunger, wherein the trigger condition is satisfied based on the motion reaching a predetermined distance.
Clause 6. The method of Clause 5, further comprising: monitoring the motion using a camera or an optical detector; and detecting that the motion reached the predetermined distance based on the camera or optical detector detecting a distance marker associated with the plunger at a predetermined location.
Clause 7. The method of any one of Clauses 1 through 6, wherein the trigger condition is triggered based on a pressure measured downstream of the syringe satisfying a pressure curve representative of how the pressure measured downstream of the syringe changes during a predetermined period at an end of the fluid delivery.
Clause 8. The method of Clause 7, further comprising: receiving an identifier associated with a type of the syringe; and performing a parameter lookup based on the identifier to obtain the trigger condition, wherein the trigger condition is determined as a result of the parameter lookup.
Clause 9. The method of Clause 7, wherein the trigger condition is determined based on a rate of the fluid delivery or historical data for one or more other fluid deliveries.
Clause 10. The method of any one of Clauses 1 through 9, wherein the syringe is coupled to the infusion device and the trigger condition is determined based on a characteristic of the syringe coupled to an infusion device.
Clause 11. The method of any one of Clauses 1 through 10, further comprising: responsive to the infusion device entering the syringe empty mode, causing a second infusion device to initiate a flush of a fluid line providing the fluid delivery from the syringe.
Clause 12. The method of any one of Clauses 1 through 11, wherein adjusting the flow rate or the threshold associated with the fluid delivery comprises: lowering the threshold, wherein the threshold is a pressure limit associated with the fluid delivery being complete, wherein the alert indicates that the syringe is empty and the fluid delivery is complete.
Clause 13. The method of any one of Clauses 1 through 12, wherein adjusting the flow rate or the threshold associated with the fluid delivery comprises: increasing a speed at which a plunger of the syringe moves to purge fluid from the syringe.
Clause 14. A non-transitory machine-readable storage medium embodying instructions that when executed by a machine, facilitate the machine to perform the method of any one of Clauses 1-13.
Clause 15. A system, comprising: one or more processors; and a memory including instructions that, when executed by the one or more processors, cause the one or more processors to perform the method of any one of Clauses 1-13.
Clause 16. An infusion device, comprising: one or more processors; and a memory including instructions that, when executed by the one or more processors, cause the one or more processors to perform the method of any one of Clauses 1-13.

### Further Consideration:

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. A method for detection and control of a syringe pump empty condition, comprising:
determining a trigger condition for entering a syringe empty mode in which an operational parameter of an infusion device is adjusted to complete a fluid delivery performed by a syringe associated with the infusion device;
monitoring the fluid delivery for the trigger condition;
responsive to the fluid delivery satisfying the trigger condition, causing the infusion device to enter the syringe empty mode and adjusting the operational parameter to complete the fluid delivery, wherein the adjusted operational parameter comprises a flow rate or a threshold associated with completing the fluid delivery;
detecting that the adjusted operational parameter has been satisfied; and
providing an alert responsive to detecting the threshold is satisfied.

2. The method of claim 1, further comprising:
monitoring a real-time delivery pressure associated with the fluid delivery; and
wherein the trigger condition is satisfied based on the real-time delivery pressure satisfying a predetermined pressure.

3. The method of claim 2, wherein the real-time delivery pressure is monitored according to a first frequency before the fluid delivery satisfying the trigger condition and increased to a second frequency responsive to the infusion device entering the syringe empty mode.

4. The method of claim 1, further comprising:
determining an amount of fluid emptied from the syringe; and
wherein the trigger condition is satisfied based on a predetermined amount of fluid emptied from the syringe.

5. The method of claim 1, wherein the syringe comprises a plunger, the method further comprising:
monitoring a motion the plunger, wherein the trigger condition is satisfied based on the motion reaching a predetermined distance.

6. The method of claim 5, further comprising:
monitoring the motion using a camera or an optical detector; and
detecting that the motion reached the predetermined distance based on the camera or the optical detector detecting a distance marker associated with the plunger at a predetermined location.

7. The method of claim 1, wherein the trigger condition is triggered based on a pressure measured downstream of the syringe satisfying a pressure curve, the pressure curve being representative of how the pressure measured downstream of the syringe changes during a predetermined period at an end of the fluid delivery.

8. The method of claim 7, further comprising:
receiving an identifier associated with a type of the syringe; and
performing a parameter lookup based on the identifier to obtain the trigger condition,
wherein the trigger condition is determined as a result of the parameter lookup.

9. The method of claim 7, wherein the trigger condition is determined based on a rate of the fluid delivery or historical data for one or more other fluid deliveries.

10. The method of claim 1, wherein the trigger condition is determined based on a characteristic of a syringe coupled to an infusion device.

11. The method of claim 1, further comprising:
responsive to the infusion device entering the syringe empty mode, causing a second infusion device to initiate a flush of a fluid line providing the fluid delivery from the syringe.

12. The method of claim 1, wherein adjusting the flow rate or the threshold associated with the fluid delivery comprises:
lowering the threshold, wherein the threshold is a pressure limit associated with the fluid delivery being complete, wherein the alert indicates that the syringe is empty and the fluid delivery is complete.

13. The method of claim 1, wherein adjusting the flow rate or the threshold associated with the fluid delivery comprises:
increasing a speed at which a plunger of the syringe moves to purge fluid from the syringe.

14. A non-transitory machine-readable storage medium embodying instructions that when executed by a machine, facilitate the machine to perform the method of claim 1.

15. A system, comprising:
one or more processors; and
memory including instructions that, when executed by the one or more processors, cause the one or more processors to perform the method of claim 1.

16. An infusion device, comprising:
one or more processors; and
memory including instructions that, when executed by the one or more processors, cause the one or more processors to perform the method of claim 1.
